(19) United States
(12) Patent Application Publication
(10) Pub. No.: US 2025/0134697 A1
(43) Pub. Date: May 1, 2025

(54) Condom-less External Male Urinary Catheter

(71) Applicants: Zachary Walter Newman, Van Nuys, CA (US); ABBIE RIVA Harooni, Tarzana, CA (US)

(72) Inventors: Zachary Walter Newman, Van Nuys, CA (US); ABBIE RIVA Harooni, Tarzana, CA (US)

(21) Appl. No.: 18/445,574

(22) Filed: Oct. 25, 2023

(51) Int. Cl. A61F 5/453; A61F 5/44; A61F 5/441

(52) U.S. Cl. CPC A61F 5/453; A61F 5/4401; A61F 5/4407; A61F 5/441

(57) ABSTRACT

A system is disclosed for collecting urine from a human male. An assembly includes absorbent material enclosed in a gusseted pouch made of polymer material. The body facing region of the Pouch is fitted with an elastomeric O ring allowing the insertion of the penis into the pouch. Once the penis is inserted into the ring, it will not retract or dislodge. The patient can urinate into the pouch while the absorbent material soaks up the discharge. The top of the pouch is fitted with a zipper that allows the changing of the pads for reuse of apparatus. The pads may be impregnated with a mild body safe deodorant or antiseptic. The Anterior face of the pouch may be fitted with a portal to accommodate a indwelling catheter and intercept the leaking urine. The pouch may have a weight to milliliter discharge conversion table, and/or a urine diagnostic strip.

### FIELD OF THE INVENTION

The present disclosure relates generally to systems, apparatus, and methods for collecting and transporting urine away from the body of a male human.

### BACKGROUND

The embodiments described herein relate generally to collecting and transporting urine away from the body of a human male. In various circumstances, a man may have limited or impaired mobility such that typical urination processes are challenging or impossible. For example, a person may experience or have a disability that impairs mobility. A person may have restricted travel conditions such as those experienced by pilots, drivers, and workers in hazardous areas. Additionally, urine collection is needed for monitoring purposes or clinical testing.

Urinary catheters, such as a Foley catheter, can be used to address some of these circumstances, such as incontinence. Unfortunately, however, urinary catheters can be uncomfortable, painful, and can lead to complications, such as infections. Prolonged use of Foley catheters can lead to a slit type rip in the penis where the Foley catheter starts leaking heavily and can no longer capture all the discharged urine leaving the patient in continuously soiled condition. The improvements herein have a solution.

Additionally, bed pans, which are receptacles used for the toileting of bedridden patients, such as those in a health care facility, are sometimes used. Bed pans, however, can be prone to discomfort, spills, and other hygiene issues. There are other external urinary catheters that can address these issues but they are also problematic. The most common type of external urinary catheter used is a condom type catheter which frequently falls off, creates lacerations on the penis making sexual intercourse painful, does not accommodate an erection, is inconvenient to use because it has to be disconnected prior to trips to bathroom at night for defecation, and finally can cause urinary tract infections similar to Foley indwelling catheters.

The second most common type of external catheter that do not use condoms are still very inconvenient to use because they require a clean shaven surface area on the pubic region of the male in order to adhere properly because they use an adhesive membrane for attachment. Furthermore, the adhesive is prone to detachment during night time sleeping and the system still relies on a tube and a collection bag at the side of the bed in order to function. This also creates the same problem as the condom type catheter where it has to be disconnected during night-trips to bathroom for the purposes of defection.

The third most popular external catheter requires a vacuum and suction and can only be used in hospital setting where a vacuum pump is readily available. There are other hair brained, bizarre contraptions on the market, but none offer a satisfactory solution. Contraptions that are bizarre or unsightly in design, can produce a negative body image. Thus, there is a need for a device capable of collecting urine from a man comfortably and with minimal contamination of the user and the surrounding area while solving the all the problems associated with the use of aforementioned Foley indwelling and external catheters all the while being comfortable to use, portable, self-contained (not requiring attachments) as well as clandestine under use.

There are hundreds of thousands of males with spinal cord injuries, many paraplegics, and quadriplegics. Most paraplegics and quadriplegics routinely rely on a care taker for diaper changes and catheter placement and emptying. Many will wait hours in urine soiled diapers and clothes before the care taker arrives to help. This improvements herein will put an end to that misery. Also Hundreds of thousands of male bedbound patients reside at nursing homes. The improvements herein have multiple benefits for them as well. If the improvements herein were to receive wide adoption by healthcare facilities, which it should because it will ultimately save them money on disposable diapers, it will greatly enhance the life quality of hundreds of thousands of patients across the country.

### SUMMARY OF THE INVENTION

A system is disclosed that is suitable for collecting and transporting urine away from the body of a human male that is portable and self-contained. The disclosed system includes an assembly that will include absorbent material enclosed in a gusseted pouch made of hydrophobic polymer material. The pouch can come in 3 different sizes. The body facing region of the Pouch is fitted with an elastomeric O ring made of polymer material for the insertion of the penis. The elastomeric O ring may come in 3 different sizes. Once the penis is inserted into the ring, it will not retract or dislodge. The patient can freely urinate into the pouch while the absorbent material soaks up the discharge through capillary action. The sides of the pouch are fitted with straps that encircle the waist for added security. The top of the pouch is fitted with a zipper that contains the unpleasant odor of the urine. The zippered top also allows for changing of pad; hence transporting the urine away from the body. The edges of the pads are impregnated with a mild body safe deodorant that will further reduce the odor of the urine. The edges of the pads may be doped with crystalline forms of chlorohexadine, sodium hypochlorite, or other body safe antiseptic. The Anterior face of the pouch may be fitted with a portal to accommodate a Foley indwelling catheter and intercept the leaking urine. The posterior or the anterior face of the pouch may have a chart converting weight of the pouch after the fluid collection to milliliters of urinary output. The interior of the pouch may be fitted with a urine diagnostic strip.

### BRIEF DESCRIPTION OF THE DRAWINGS

FIG. 1 is an anterior view of a urine collection device.

FIG. 12 is an anterior perspective view of the urine collection device of FIG. 1.

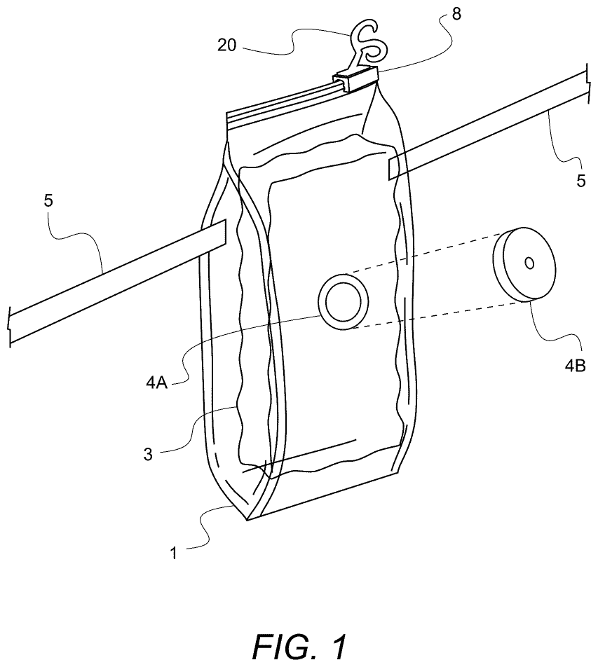

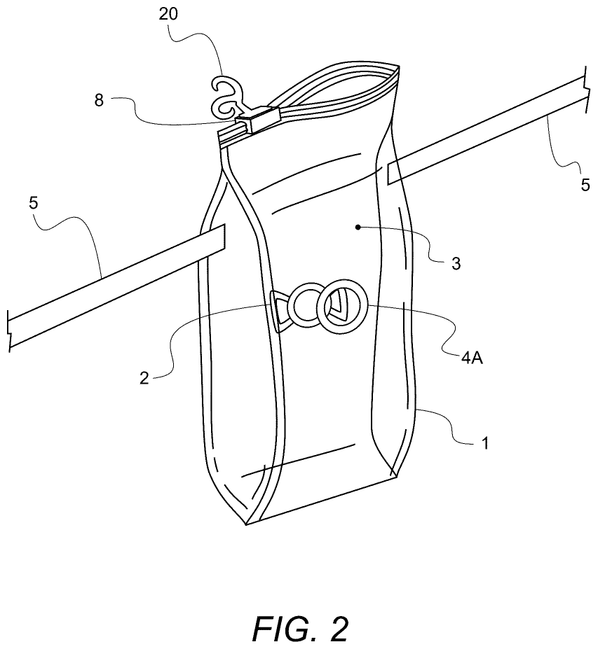

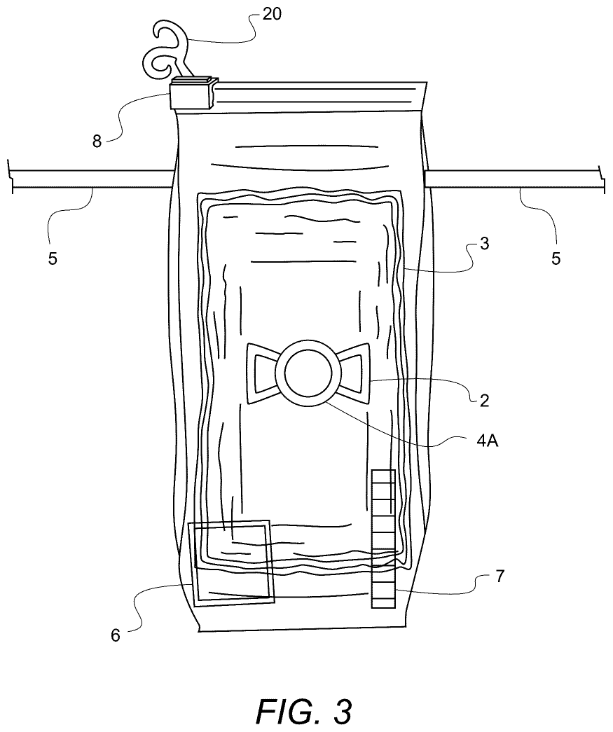

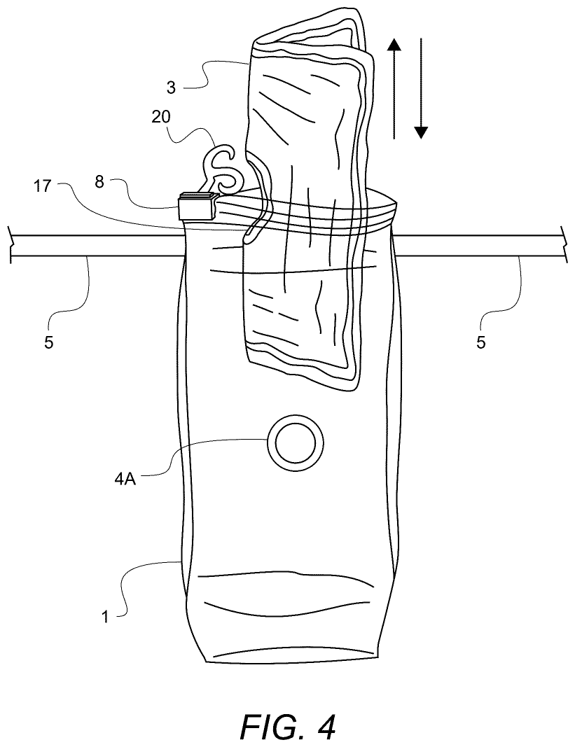

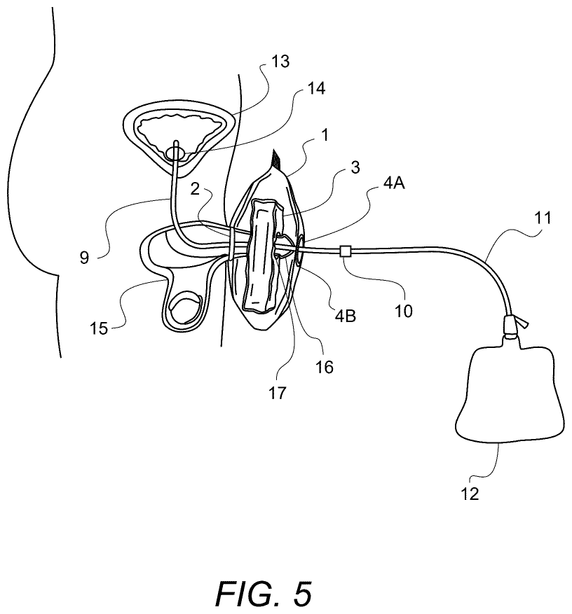

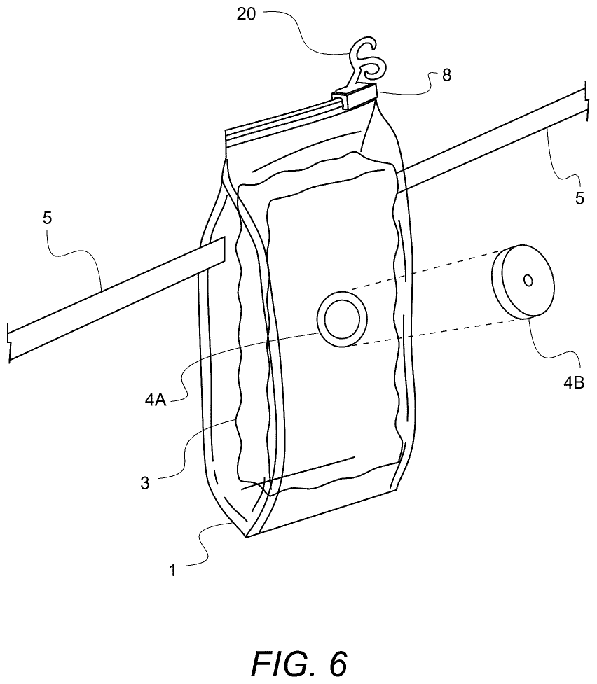

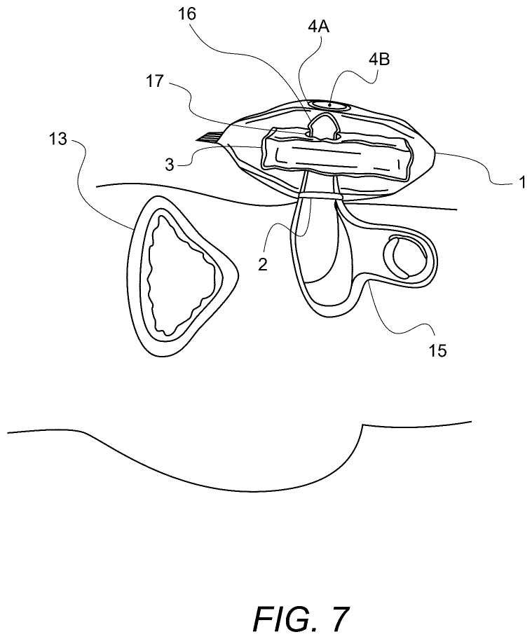

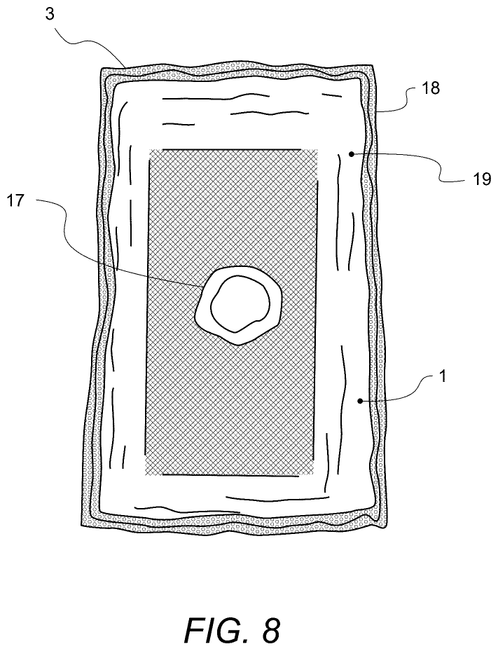

FIG. 3 is a posterior (patient facing) view of the urine collection device of FIG. 1.

FIG. 4 depicts insertion of the pad inside the pouch of the urine collection device of FIG. 1.

FIG. 5 shows the urine collection device of FIG. 1, in use in conjunction with an indwelling Foley catheter for a male patient in supine position. #

FIG. 6 is an anterior view of the urine collection device of FIG. 1.

FIG. 7 depicts us of the urine collection device of FIG. 1 for a male patient in a supine position.

FIG. 8 is a cross-sectional view of the pad inside the pouch of the urine collection device of FIG. 1.

### DETAILED DESCRIPTION

The following discussion provides many example embodiments of the inventive subject matter. Although each embodiment represents a single combination of inventive elements, the inventive subject matter is considered to include all possible combinations of the disclosed elements. Thus if one embodiment comprises elements A, B, and C, and a second embodiment comprises elements B and D, then the inventive subject matter is also considered to include other remaining combinations of A, B, C, or D, even if not explicitly disclosed.

As used herein, and unless the context dictates otherwise, the term “coupled to” is intended to include both direct coupling (in which two elements that are coupled to each other contact each other) and indirect coupling (in which at least one additional element is located between the two elements). Therefore, the terms “coupled to” and “coupled with” are used synonymously.

A system is disclosed that is suitable for collecting and transporting urine away from the body of a human male that is portable and self-contained. The disclosed system includes an assembly that will include absorbent material for the transportation of urine enclosed in a gusseted pouch made of hydrophobic polymer material for the collection of urine. The pouch can come in 3 different sizes. In one implementation, pouch 1, FIG. 1 1 may be 4 inches by 6 inches. This system is designed to collect urine for 5 hours for patients that are only suffering from mild dripping type of urine discharge. On a second implementation, pouch 1 FIG. 1 1 may be 6 inches by 9 inches. This implementation is designed to collect urine for up to 9 hours on a low discharge level and up to 4 hours for a normal Urinary output level of 80 milliliters per hour. On a third implementation, pouch 1 FIG. 1 may be sized at 9 inches by 11 inches. This system is designed to replace an adult diaper and can collect up to 12 hours of urine at normal human urinary output level of 80 ml/hr. All three implementations are suitable for 24 hour use, awake or sleep (e.g., FIG. 2) ambulatory or non-ambulatory.

FIG. 1 shows the top of the pouch 1 is fitted with zipper closure 8 (alternatively a flap type closure) in order to control and reduce the dispersion of the urine odor into the patients' surroundings. Handle 20 of the zipper closure 8 has a large bidirectional S shaped handle suitable for paraplegics and quadriplegics with limited use of their upper digits.

Interior pad 3 of FIGS. 1-8 of the urine collection device is constructed of absorbent material, woven or non-woven, trimmed to fit inside the pouch, for example pouch 1 of FIGS. 1-8 1, 12, and 4. The interior pads 3 have hole 17 in the middle to receive the penis. (FIGS. 4 and 8) The edges of the pads may be impregnated by a body safe deodorant material 18, (FIG. 8) to neutralize the odor associated with Urine. See FIG. 3. The deodorant impregnated portion 18 6 of interior pad 3 does not come in contact with the penis until a urination event. This is an extra safety precaution. In some embodiments, the edges of pouch 1 are doped with body safe antiseptic materials 19 to disinfect the urine of patients with a history of urinary tract infections (FIG. 8) 3. This urine collection device is novel and different from a regular absorbent pad. The edges of pouch 1 are doped with antiseptic crystals 19 to prevent direct contact with the penis. Only after a urination event will the crystals dissolve and sanitize the discharged urine. The amount of antiseptic crystals will be adjusted in such way as to create the right aqueous concentration of the antiseptic in order to be body safe. Such disinfectants may be chlorohexadine crystals or sodium Hypochlorite microencapsulation. Safer and more user friendly antiseptics can always be substituted.

In FIG. 2, pouch 1 is fitted with elastomeric O ring 2, made of polymers, that expands in order to receive the penis, as seen in FIG. 3. O ring 2 is attached to the pouch via heat sealing, creating a leak proof seal. Once the Penis is inserted, O ring 2 contracts spontaneously to form a snug fit around the penis. O ring 2 should not be confused with just a simple hole in order to insert the penis within the receptacle/collection device. O ring 2 is more than just a hole, it's a device all on its own with many functions that will be discussed. O ring 2 is a critical element of the design for a number of reasons.

First, O ring 2 prevents backflow of urine out of the pouch; hence the apparatus can be used in a supine, Prone, or sims positions while awake or during sleep. It achieves this functionality by contracting in order to form a tight fit around the penis. Secondly O ring 2 aids to hold the pouch in place, again by its capability to contract to form fit around the penis. Third, O ring 2 expands to accommodate an erection and subsequently spontaneously contracts to reform the snug fit once the erection has subsided. Fourth, O ring 2 holds the penis in place and does not allow it to retract.

Many current catheter designs will fail during backlying (supine), side lying (sims) positions, during erections and retractions. The improvements herein allows erections, allow use during sleep, prevents retractions, all the while keeping the catheter in place, functioning properly. Alternative designs known today require a vacuum pump, are not self-contained, are only available at hospital settings, and it are not portable, and further require use of adhesives to secure the catheter to the body of the patient. As such, if the patient's body parts are not clean shaven or the adhesives fails, so will the whole system. The use of adhesives may also irritate the skin over time. The Adhesive securing the catheter in place will eventually fail after a few days of hair growth underneath the contact area.

The improvements herein do not require adhesives, vacuum pumps, and shaving the areas around the genitals to function properly—the improvements herein appear novel. Improvements herein enable portability and can be used by ambulatory patients as well as general public.

Finally and perhaps most importantly, O ring 2 has minimal surface contact with the penis. This design minimizes possible irritation to the skin of the penis. Other designs such as the condom catheter or bag on penis catheter or other designs using a sheath over the penis, have the potential, and do in fact irritate the skin of the penis to the point of lacerations. O ring 2 is only in contact with 3% of the penis skin surface area while other sheath and non-sheath type catheters may cover anywhere from 20% to 100% percent of the penis surface area. The more surface area contact with the penis, the greater the chance of lacerations. An intact penis surface skin is a crucial benefit for sexually active paraplegics, quadriplegics or anyone for that matter. No known designs address this very important issue. O ring 2 may be available in three or more different sizes in order to accommodate different size penises.

The sides of pouch 1 are optionally fitted with straps 5 that encircle the waist for added security in holding the catheter in place, as seen in FIGS. 1-6) 1, 12, 3, 4, and 6.

The anterior facing (away from the patient) side of the catheter, is fitted with portal 4A (see FIGS. 1-7)) to receive indwelling Foley type catheter 9 of FIG. 5. The embodiments herein are the first known to incorporate such feature. This Embodiment is specifically designed for patients who have suffered from urinary retention for many years and have been using Foley type catheters for years. Foley type catheters, with long term use, start leaking heavily. It eventually gets to a point that the Foley catheter can only remove ½ of the urine discharge. The other half of the urine discharge simply leaks all over the patients' bed or clothing. A diaper cannot be used in conjunction with the Foley catheter because the Foley catheter is in the way.

The improvements herein solve that problem by creating portal 4 through the pouch, where the leaking urine can be collected the disclosed apparatuses. This is achieved easily by passing the penis 16, penis shaft 15, and the Foley catheter 9 into O ring 2 receptacle from one end and the Foley out through anterior portal 4A of the apparatus, with the Foley extending out towards its tubing 10 and 11, and finally to its designated urinary collection bag 12 as in FIG. 5. In this configuration, any urine that is not collected by the Foley, will be collected by the disclosed apparatuses, leaving the patient clean, dry, comfortable, free from skin irritation and pressure sores. Portal 4A can simply be closed by cap 4B when not in use, as in FIGS. 1,5,6 and 7) 1.

Our design also has other medically important features. First, the posterior or anterior face of pouch 1 is furnished with a weight-to-urinary-output conversion chart 6 as in FIG. 3 3. This is very useful because when the catheter is being used as an alternative to diapers in a healthcare setting, the urinary output of the patient can be measured very accurately. Healthcare facilities commonly measure the amount of urinary output using the number of diaper changes per shift, which is grossly inaccurate. With our design, pouch 1 can simply be weighed on a scale and the conversion table on the pouch will alert the nurse/caretaker to the exact amount of urinary output.

The diagnostic capabilities of the apparatus is further enhanced by the incorporation of urinary diagnostic strip 7 inside pouch 1 as in FIG. 3. Strip 7 has the capability to alert the nurse to conditions such as proteinuria, dehydration, and possible urinary tract infections.

Another advantageous feature of our catheter design is that it is reusable. Pads 3 inside the pouch can be replaced as long as the exterior encapsulating pouch 1 is intact and functional. Most known catheters such as the condom catheter, bag over penis catheter, other sheath type catheters, as well as the vacuum assisted urinary catheter contain parts that are not reusable. Each part of the disclosed embodiments, with the exception of the pouch 1 and the interior pads 3, are optionally reusable. This creates tremendous cost savings to individuals, healthcare facilities and the environment in general. Interior pads 3 are optionally biodegradable.

A key feature of improvements herein is that the urine collection device it can be used as a diaper at home, on the road, as well as healthcare setting due to its portability and Self-containment. The catheter is suitable for bedbound patients as well as Paraplegics, and quadriplegics because it uses capillary action of the absorbent material to collect the urine while the patient is at fowlers, supine, prone, or sims positions. No known catheter provides this function without a vacuum pump. Even vacuum assisted catheters restrict the movement of the bedbound patients.

This feature is important for a number of reasons. First it allows for the timely repositioning of the bedbound patient during day or night. Most doctors' orders require the repositioning of the bedbound patients every 2 hours in order to prevent bedsores. Secondly, nursing homes are often understaffed and patients have to wait for hours for a diaper change. Meanwhile the skin of the patient is in contact with the wet diaper, slowly being dissolved. Our apparatus keeps the urine away from the surface of the skin for up to 12 hours need be. This feature will greatly reduce the number of skin breakdowns associated with prolonged urine-skin contact. It also has the added benefit of freeing the hands of personnel performing diaper changes, in order to perform less diaper changes and more patient repositioning. This arrangement ultimately will greatly reduce the number of bedsores in facilities and the subsequent wound care needs associated with it. Bed sore wounds are anywhere from unsightly and malodorous to downright painful at the beginning of their formation, not to mention that their treatment costs taxpayers multi-millions of dollars per year. Every healthcare facility has 2-4 nurses specifically dedicated to treating pressure sore wounds.

Examples of polymers include nylons, polyesters, polycarbonates, polyacrylates, polymers of ethylene-vinyl acetates and other acyl substituted cellulose acetates, non-degradable polyurethanes, polystyrenes, polyvinyl chloride, polyvinyl fluoride, poly (vinyl imidazole), chlorosulphonate polyolefins, polyethylene oxide, polyethylene terephthalate (PET), polytetrafluoroethylene (PTFE), and/or blends and copolymers thereof.

It should be apparent to those skilled in the art that many more modifications besides those already described are possible without departing from the inventive concepts herein. The inventive subject matter, therefore, is not to be restricted except in the spirit of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Where the specification claims refers to at least one of something selected from the group consisting of A, B, C . . . and N, the text should be interpreted as requiring only one element from the group, not A plus N, or B plus N, etc.

All publications herein are incorporated by reference to the same extent as if each individual publication or patent application were specifically and individually indicated to be incorporated by reference. Where a definition or use of a term in an incorporated reference is inconsistent or contrary to the definition of that term provided herein, the definition of that term provided herein applies and the definition of that term in the reference does not apply.

The recitation of ranges of values herein is merely intended to serve as a shorthand method of referring individually to each separate value falling within the range. Unless otherwise indicated herein, each individual value is incorporated into the specification as if it were individually recited herein, and ranges include their endpoints.

In some embodiments, the numbers expressing quantities of ingredients, properties such as concentration, reaction conditions, and so forth, used to describe and claim certain embodiments of the invention are to be understood as being modified in some instances by the term “about.” Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable. The numerical values presented in some embodiments of the invention may contain certain errors necessarily resulting from the standard deviation found in their respective testing measurements.

As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise.

All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the invention and does not pose a limitation on the scope of the invention otherwise claimed. No language in the specification should be construed as indicating any non-claimed element essential to the practice of the invention. Unless a contrary meaning is explicitly stated, all ranges are inclusive of their endpoints, and open-ended ranges are to be interpreted as bounded on the open end by commercially feasible embodiments.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

1. A urine collection device, comprising:
a portable collection chamber; and
a penis receptacle fitted into a first wall of the portable collection chamber.

2. The device of claim 1, wherein the portable collection chamber or the penis receptacle is at least 90% biodegradable, either alone or in combination.

3. The device of claim 1, wherein the first wall of the portable collection chamber comprises a waterproof polymer.

4. The device of claim 1, further comprising an absorbent pad disposed within the portable collection chamber, wherein one of a deodorant crystal or a deodorant compound is disposed at an edge of the absorbent pad.

5. The device of claim 1, further comprising an absorbent pad disposed within the portable collection chamber, wherein one of an antiseptic crystal or an antiseptic compound is disposed at an edge of the absorbent pad.

6. The device of claim 1, further comprising a one of a semi-airtight zipper or a flap type closure disposed on a second wall of the portable collection chamber.

7. The device of claim 1, wherein an edge of the penis receptacle comprises an elastomeric material configured to contract about a member present in the penis receptacle.

8. The device of claim 7, wherein the elastomeric material expands or contracts in correspondence with a change of a dimension of the member at the penis receptacle.

9. The device of claim 7, wherein the elastomeric material forms a fluidic seal about the member present in the penis receptacle, thereby preventing leaking of fluid from the portable collection chamber.

10. The device of claim 7, wherein the portable collection chamber further comprises a hole configured to receive a portion of a catheter from outside of the portable collection chamber.

11. The device of claim 7, wherein the elastomeric material is in direct contact with less than 5% of a surface area of the member.

12. A system of holding a catheter in place with respect to a penis, comprising:
an elastomeric O ring coupled to the catheter, wherein the O ring is configured to contract around a penis and to restrict a movement of the penis away from the O ring.

13. The system of claim 12, wherein the O ring forms a fluidic seal about the penis, thereby preventing leaking of urine from the catheter

14. The system of claim 12, wherein the O ring expands or contracts in correspondence with a change of a dimension of the penis at the penis receptacle.

15. The system of claim 12, wherein the O ring is in direct contact with less than 5% of a surface area of the member.

16. The system of claim 12, wherein the catheter further comprises a hole configured to receive a portion of a Foley catheter.
